(19) United States
(12) Patent Application Publication
(10) Pub. No.: US 2006/0161267 A1
(43) Pub. Date: Jul. 20, 2006

(54) Method and apparatus for applying mirror-printed film to a prosthetic or orthotic device and device having the same

(76) Inventor: Arinbjorn V. Clausen, Reykjavik (IS)

Correspondence Address: KNOBBE MARTENS OLSON & BEAR LLP, 2040 MAIN STREET, FOURTEENTH FLOOR, IRVINE, CA, 92614, US

(21) Appl. No.: 11/073,037

(22) Filed: Mar. 4, 2005

Related U.S. Application Data
(60) Provisional application No. 60/644,133, filed on Jan. 14, 2005.

(51) Int. Cl. A61F 2/66; A61F 5/052; B32B 31/12

(52) U.S. Cl. 623/55; 602/6; 264/173.11

(57) ABSTRACT

A device for attaching to a limb comprises multiple layers of shapeable material having a curved configuration, said multiple layers together defining a first and second surface of said device, and a film having at least one mirror-printed image thereon adhered to at least one of said first and second surfaces of the device.

### RELATED APPLICATION

This application claims priority to U.S. Provisional Application No. 60/644,133, filed Jan. 14, 2005, the entire contents of which are hereby incorporated by reference and should be considered a part of this specification.

### FIELD OF THE INVENTION

The present embodiments generally relate to prosthetic or orthotic devices, and more particularly, to a method and apparatus for applying a film to a prosthetic or orthotic device and to devices having the same.

### DESCRIPTION OF THE RELATED ART

Various designs for orthotic and prosthetic devices exist in the art. Known methods for manufacturing such devices are also known in the art. However, such devices cause wear and tear in related products used with the devices, such as covers, due to sharp edges on rough surfaces on the devices. Additionally, conventional prosthetic and orthotic devices are subject to vibration forces during use, which are uncomfortable to a user of such a device and wears down the device. Accordingly, a need exists for an improved prosthetic or orthotic device and a method for manufacturing the same.

### SUMMARY OF THE INVENTION

In accordance with one embodiment of the invention, a device for attaching to a limb is provided. The device comprises multiple layers of shapeable material having a curved configuration, said multiple layers together defining a first and second surface of the device. The device also comprises a film having at least one mirror-printed image thereon adhered to the at least one of said first and second surfaces of the device.

In accordance with another embodiment of the invention, a prosthetic device is provided. The prosthetic device comprises a foot plate having an upper portion and a lower portion, wherein at least a portion of the foot plate extends generally downward and forward, said foot plate having first and second surfaces. The prosthetic device also comprises an adapter provided adjacent the upper portion of the foot plate and configured to attach said foot plate to a pylon or other leg prosthesis. The prosthetic device also comprises a film applied to at least one of the first and second surfaces, the film having a bottom surface adhered to at least one of the upper and lower surfaces and having a mirror-printed image provided thereon facing at least one of the first and second surfaces.

In accordance with yet another embodiment of the invention, a prosthetic device is provided comprising one or more foot plates. At least one of said one or more foot plates has an upper portion at about a location along a natural human lower leg and at least one of said one or more foot plates has a lower portion at about a location along a natural human sole, said one or more foot plates each having opposing surfaces. The prosthetic device also comprises an adapter provided adjacent the upper portion and configure to attach said one or more foot plates to a pylon or other leg prosthesis. The prosthetic device also comprises a film applied to at least one of the opposing surfaces. The film has a bottom surface adhered to at least one of the opposing surfaces and has a mirror-printed image provided thereon facing at least one of the opposing surfaces.

In accordance with still another embodiment of the invention, a method for manufacturing a device that attaches to a limb is provided. The method comprises providing a film having at least one mirror-printed image thereon and providing a plurality of layers of shapeable material to form a device for attaching to a limb. The method also comprises applying said film to at least one of said layers of shapeable material with the mirror-printed image facing the at least one layer.

### BRIEF DESCRIPTION OF THE DRAWINGS

FIG. 1 is a perspective schematic view of one embodiment of a mirror-printed film.

FIG. 2 is a top schematic view of the mirror-printed film of FIG. 1.

FIG. 3 is a perspective schematic view one embodiment of a tool for forming a shaped article.

FIG. 4 is a flow-chart illustrating one embodiment of a method for applying a mirror-printed film.

FIG. 5 is a flow-chart illustrating another embodiment of a method for applying a mirror-printed film.

FIG. 6A is a perspective schematic view of one step of a method for applying a mirror-printed film.

FIG. 6B is a top schematic view of another step of a method for applying a mirror-printed film.

FIG. 6C is a top schematic view of another step of a method for applying a mirror-printed film.

FIG. 6D is a top schematic view of another step of a method for applying a mirror-printed film.

FIG. 6E is a top schematic view of another step of a method for applying a mirror-printed film.

FIG. 6F is a top schematic view of another step of a method for applying a mirror-printed film.

FIG. 6G is a top schematic view of another step of a method for applying a mirror-printed film.

FIG. 7 is a cross-sectional schematic view of a completed construct produced according to one or a combination of the methods illustrated in FIGS. 3 and 4.

FIG. 8 is a profile schematic view of a shaped article having a mirror-printed film.

### DETAILED DESCRIPTION OF THE PREFERRED EMBODIMENTS

Briefly stated, the preferred embodiments hereinbelow describe a method for applying a mirror-printed film to a shaped article during the manufacture of said article. Such articles can include a prosthesis or prosthetic device, and an orthosis or orthotic device.

The term “orthotic” and “orthosis” as used herein are broad terms and are used in their ordinary sense and refer to, without limitation, any system, device or apparatus that may be used to support, align, prevent, protect, correct deformities of, immobilize, or improve the function of parts of the body, such as joints and/or limbs.

The terms “prosthetic” and “prosthesis” as used herein are broad terms and are used in their ordinary sense and refer to, without limitation, any system, device or apparatus that may be used as an artificial substitute or support for a body part. A prosthesis or prosthetic device can include, without limitation, foot plates, heel plates, ankle plates, and lower leg pylons. Additionally, prosthetic devices may be curved to approximate a joint of a human limb and can have, without limitation, the following shapes alone or in combination: J-shape, L-shape, C-shape, U-shape, S-shape, Z-shape and upside down Y-shape.

The shaped article is made of shapeable materials. For example, the shaped article can include fiber-reinforced resin composite materials. Various fiber-reinforced materials include, but are not limited to, continuous fibers of glass, aramid, graphite, etc., and resins of epoxy, vinyl ester, thermoplastics, cyanate ester, polyester, polyurethane, and thermoset acrylic. Fibers may be continuous or discontinuous, aligned or random, woven or non-woven. It is also conceivable to include layers of metals, plastic, film adhesive, sheet foam, syntactic foam, or other lightweight core materials. Furthermore, although the embodiments particularly describe the construction of prosthetic feet, the method of applying said film can be applied to the construction of other prosthetic devices as well as any article having a desired shape. The mirror-printed film protects and improves the operation of the shaped article, as discussed below.

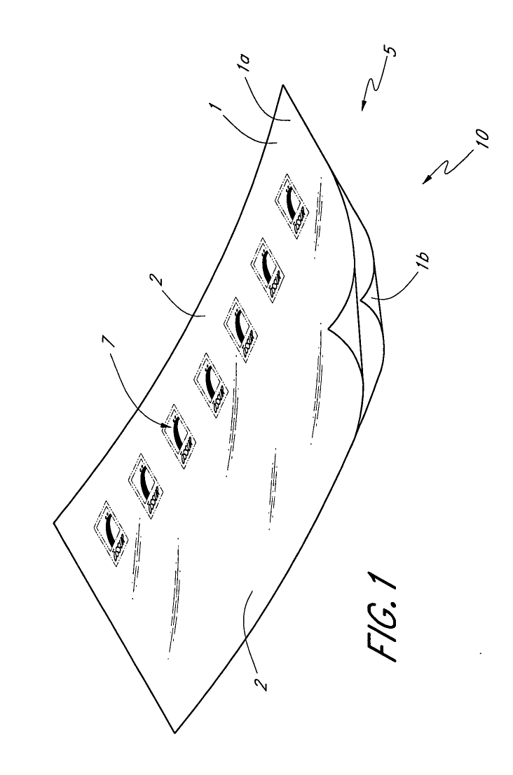

FIG. 1 shows one embodiment of a mirror-printed film 10. The mirror-printed film preferably comprises a first film 1, having a top surface 1a and an opposite bottom surface 1b. In a preferred embodiment, the bottom surface 1b of the first film 1 is treated to maximize bonding of the first film 1 to the shapeable materials that make up the shaped article, as discussed further below. In one embodiment, the first film 1 can be sanded and flame treated to maximize bonding of the first film 1 to the shapeable materials. A second film 2 is preferably removably attached to the top surface 1a of the first film 1, and together with the first film 1 constitutes a film assembly 5. Advantageously, the second film 2 protects the top surface 1a of the first film 1 from, for example, scratching.

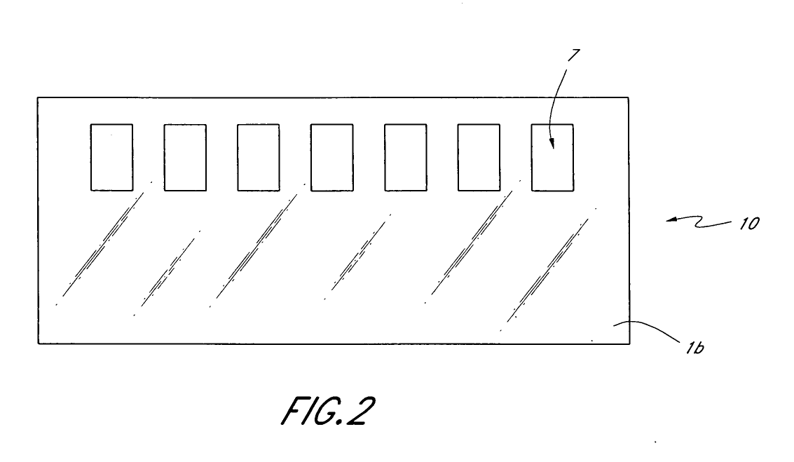

As shown in FIG. 1, the first film 1 is preferably a clear film. In another embodiment, the first film 1 can be substantially translucent. In still another embodiment, the first film 1 can be opaque. The mirror-printed film 10 can have at least one graphic 7 printed in reverse (i.e., mirror-printed) on the bottom surface 1b of the first film 1 (see FIG. 2). Such mirror printing allows the graphic to be viewed right-side up when viewed from the top surface 1a of the first film 1. In the illustrated embodiment, a plurality of repeating graphic images 7 have been printed on the bottom surface 1b of the first film 1, as seen in FIG. 1. One of ordinary skill in the art will recognize that the mirror-printed film 10 can have any number of graphic images 7 printed thereon. Where the mirror-printed film 10 has multiple images 7, the images may all be of the same design or can each be of different designs. Additionally, one of ordinary skill in the art will recognize that any number of graphic designs, patterns and colors can be mirror-printed on the first film 1. In a different embodiment, the first film 1 can be used without mirror printing a graphic on it. In still another embodiment, the first film 1 can be a colored film.

The first film 1 is preferably made of a flexible and resilient material that is UV-radiation resistant and impact resistant. Additionally, the first film 1 can preferably dampen vibration forces, and has a thickness suitable to reduce the wear and tear of the shaped article. In one embodiment, the first film 1 can be made of a thermo-plastic urethane (TPU) material. In another embodiment, the first film 1 can be made of an acrylnitrile-butadiene-styrene (ABS) copolymer material. In still another embodiment, the first film 1 material can be an ABS/TPU blend. In yet another embodiment, the first film 1 can be made of a polyamid material. In one embodiment, the second film 2 is made of the same material as the first film 1. In another embodiment, the second film 2 can be made of a plastic material.

In a preferred embodiment, the first film 1 has a density of between about 1 g/cm³ and about 1.2 g/cm³, and more preferably between about 1.1 g/cm³ and about 1.13 g/cm³. The first film 1 also preferably has a thickness of between about 0.3 mm and about 2.5 mm, and more preferably between about 0.3 mm and about 2.2 mm. Additionally, in a preferred embodiment, the first film 1 has a modulus of elasticity of between about 300 MPa and about 2000 MPa, and more preferably between about 500 MPa and about 1600 MPa. Further, in a preferred embodiment, the first film 1 has a tensile strength at break of between about 30 MPa and about 75 MPa, and more preferably between about 33 MPa and about 39 MPa. Additionally, the first film 1 preferably can withstand temperatures less than about 20° C., as well as temperatures greater than about 100° C. One such suitable material for the first film 1 is the Duraclear® product supplied by IMS AG of Austria.

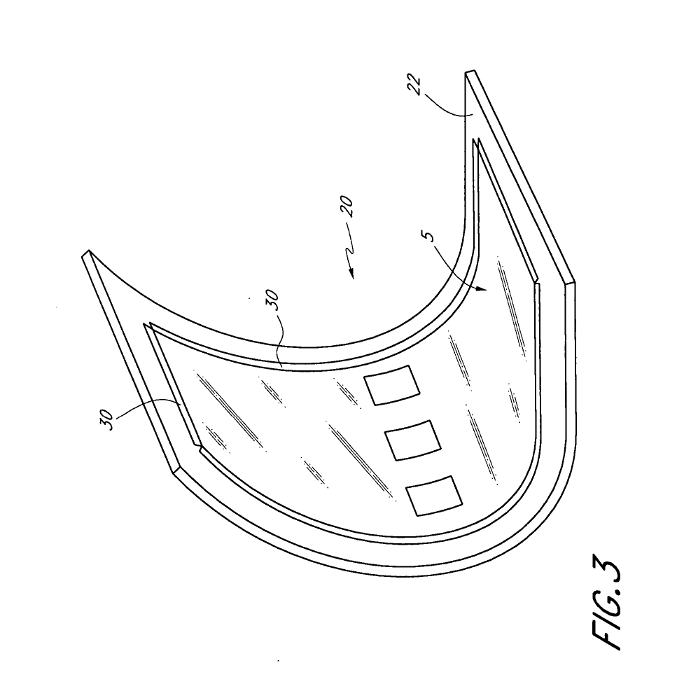

FIG. 3 shows one embodiment of a tool 20 used to form the shaped article. In a preferred embodiment, the tool 20 is a mold to which the shapeable material is applied in layers or panels. The tool 20 is preferably curved. A film assembly 5, comprising the first 1 and second 2 films, is removably fixed onto a top surface 22 of the tool 20 so that the second film 2 is adjacent the top or contoured surface 22 of the tool and the bottom surface 1b of the first film 1 faces away from the surface 22 of the tool 20. As discussed previously, in one embodiment, the first film 1 can have a mirror-printed image 7 thereon. In another embodiment, the first film 1 can be a film without any images printed thereon. As shown in FIG. 3, tape 30 can be used to fix the film assembly 5 to the top surface 22 of the tool 20. In the illustrated embodiment, the film assembly 5 is taped to the tool 20 along substantially the entire length of the edges of the film assembly 5. In other embodiments, the film assembly 5 can be removably fixed to the top surface 22 of the tool 20 via other mechanisms, such as an adhesive.

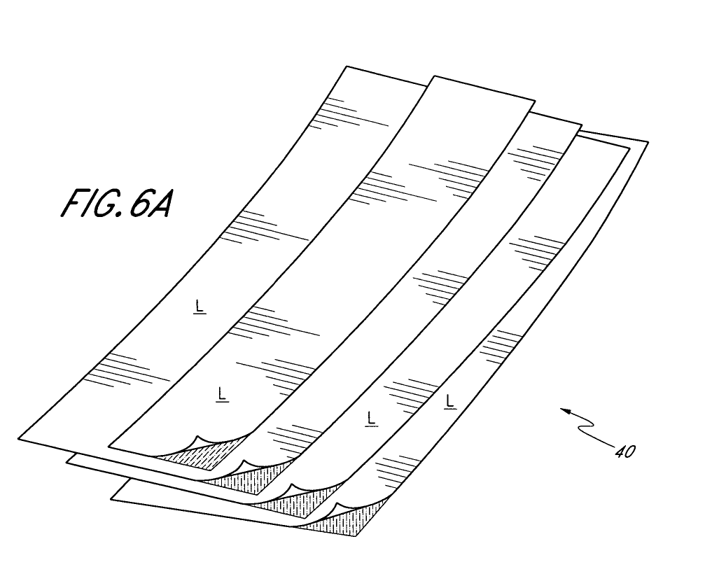

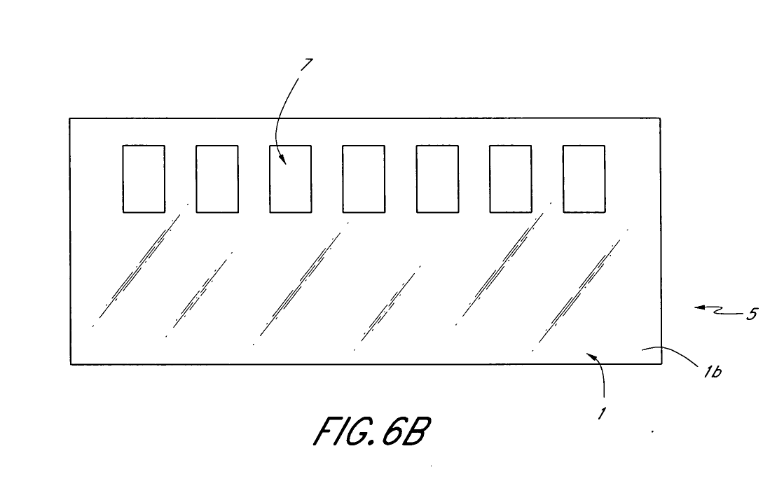

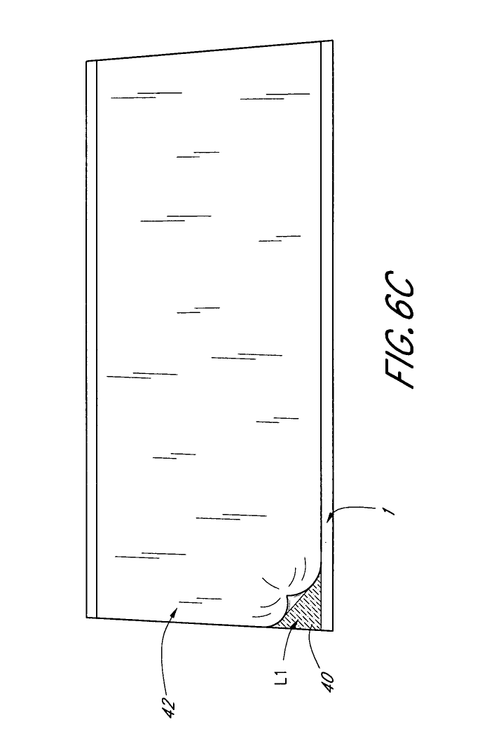

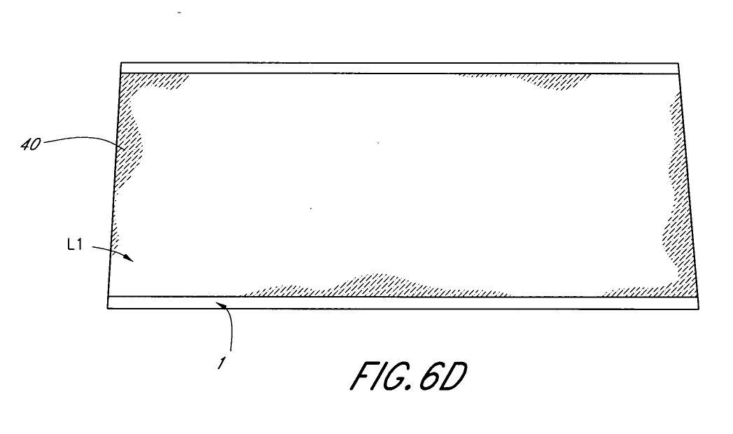

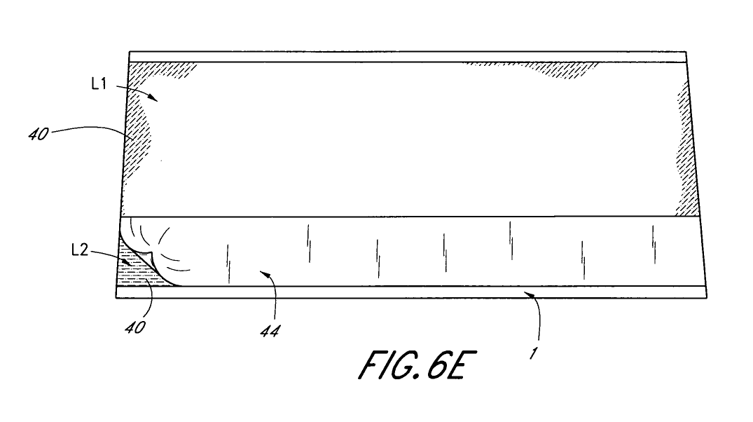

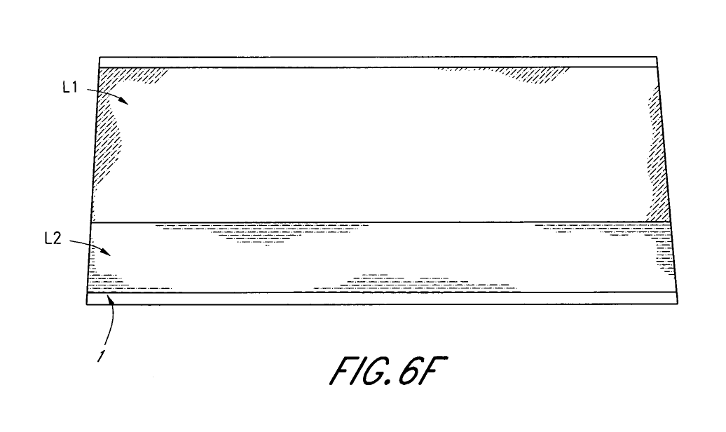

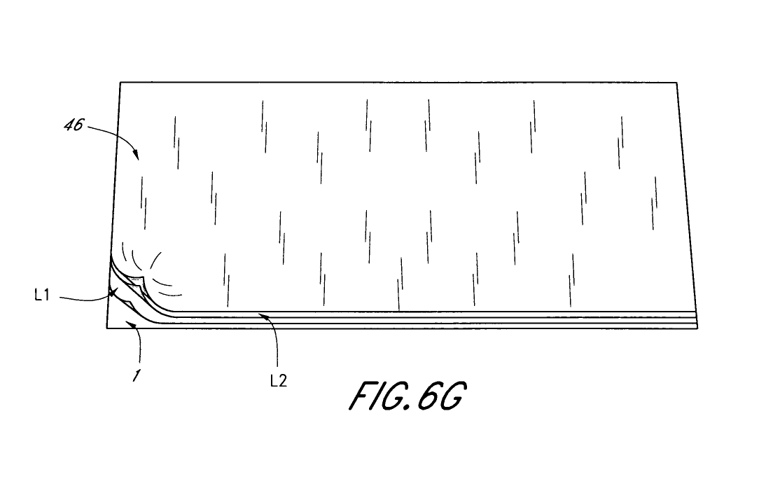

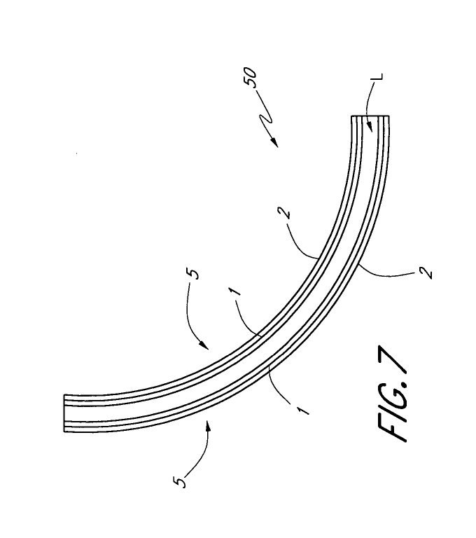

After the film assembly 5 is fixed to the top surface 22 of the tool 20, at least one layer L of shapeable material 40 (see FIG. 6A) can then be applied on top of the first film 1 to form a construct 50 of shapeable material 40 (see FIG. 7). That is, the bottom surface 1b of the first film 1, on which the at least one graphic 7 has been printed, is placed in contact with a layer L of shapeable material 40. The at least one layer L of shapeable material 40 can include a combination of layers L of different materials, such as those listed above, and are applied to the tool using a desired lay-up sequence. Further description of lay-up sequences can be found in U.S. patent application Ser. No. 10/944,436, filed May 28, 2004, the contents of which are hereby incorporated in their entirety. In a preferred embodiment a second film assembly 5 is placed on top of the last of the layers L of shapeable material 40 so that the bottom surface 1b of its first film 1 contacts said last layer L of shapeable material 40 and its second film 2 faces away from the construct 50 of shapeable material 40.

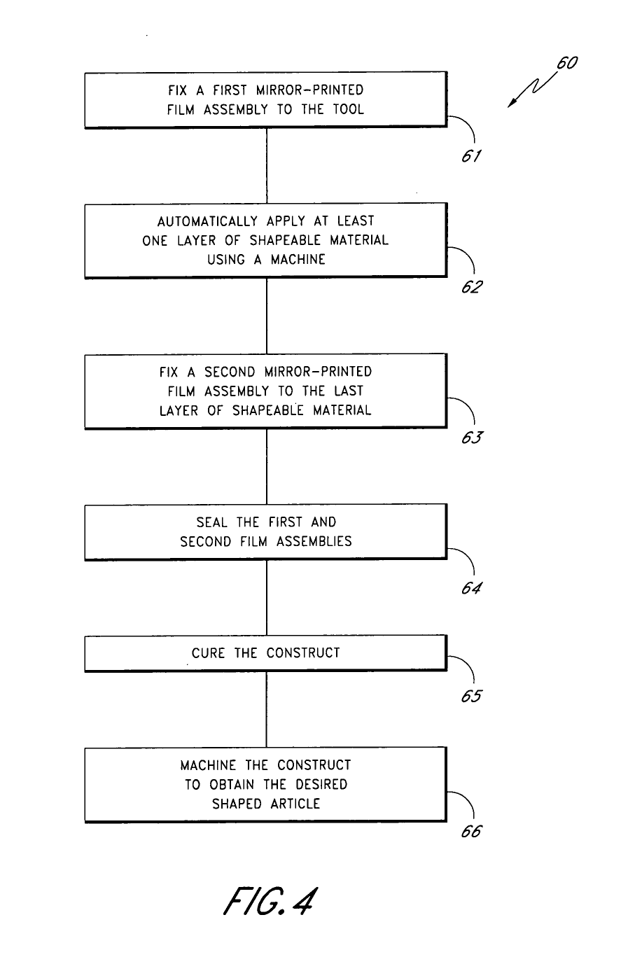

One embodiment of a method 60 for manufacturing the shaped article with a mirror-printed film thereon is detailed in FIG. 4. As noted above, the method includes fixing 61 a first film assembly 5 onto the tool 20, as shown in FIG. 3.

In the embodiment illustrated in FIG. 4, at least one layer L of shapeable material 40 is then automatically applied 62 to the tool 20 using a machine. The machine preferably applies pressure to each layer L or panel as it is positioned adjacent the previous layer L, in order to substantially bond the layers together. In one embodiment, about thirty layers L of carbon fiber are applied. However, other types of shapeable material 40, and any suitable number of layers L can be applied to form the construct 50 of shapeable material 40. Preferably, each layer L of shapeable material 40 has an epoxy resin on at least one surface of the layer, the epoxy configured to substantially bond the layers together.

As detailed in FIG. 4, once the last layer L of shapeable material 40 has been applied, a second film assembly 5 is applied 63 to the last layer L so that the bottom surface 1b of its first film 1 faces the construct 50 and its second film 2 faces away from the construct 50. The first and second film assemblies 5 are then sealed 64 along the sides of the construct 50. In a preferred embodiment, the film assemblies 5 are sealed with tape. In other embodiments, the film assemblies 5 can be sealed in other suitable manners, such as with an adhesive. In one embodiment, the first film 1 of at least one of the first and second film assemblies 5 has a mirror-printed image 7 on the bottom surface 1b thereof. In another embodiment, the first film 1 on both of the first and second film assemblies 5 has a mirror-printed image 7 on the bottom surface 1b thereof.

The construct 50 is then cured 65. Preferably, the construct 50 is cured in a pressurized autoclaved oven (not shown) while a vacuum is applied to withdraw air from the construct 50. In one embodiment, a pressure of about 7 bars is applied during the curing process. The construct 50 is preferably cured as required by the manufacturer of the epoxy resin to increase the adherence of the layers of shapeable material and mirror-printed films 5 to each other. Accordingly, the construct 50 is cured to optimize the bonding properties of the epoxy resin. In one embodiment, where the epoxy resin is model number NewportBond 301 supplied by Newport Adhesive and Composites, Inc. of Irvine, Calif., the construct 50 is cured to a temperature in a range between about 107° C. and about 150° C.

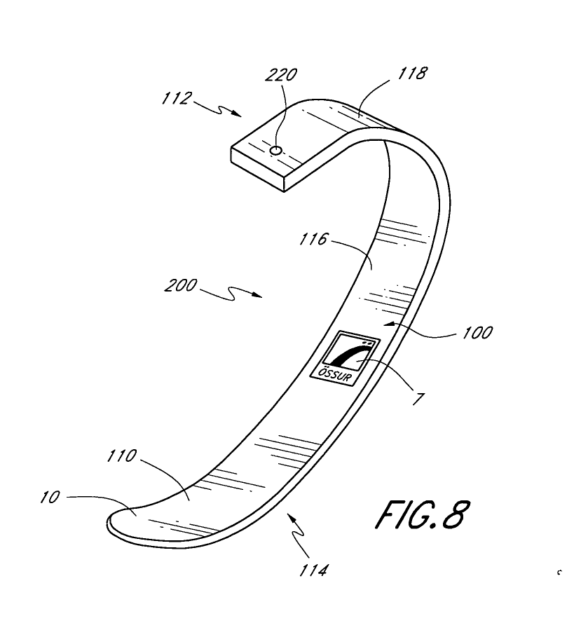

In the embodiment illustrated in FIG. 4, after the construct 50 is cured, the construct can be machined 66 to obtain the shaped article 100, which can be a prosthetic device (see FIG. 8) or an orthotic device for attaching to a limb. For example, a cutting device (not shown) can be used to machine the construct 50 into the desired shape of the shaped article 100. As noted above, the mirror-printed film 10 can have multiple graphic images 7 printed thereon. Accordingly, the cutting device can be used to machine the construct 50 in order to obtain multiple shaped articles 100 from the construct 50, each article having one of the graphic images 7 thereon. In another embodiment, each of the shaped articles 100 can have more than one of the graphic images 7 thereon. Therefore, a mirror-printed film 10 with multiple graphic images 7 printed thereon can be used to mass label shaped articles that are machined from a completed construct 50. In one embodiment, the cutting device is a water jet (not shown). However, any suitable cutting device can be used. Once the shaped article 100 is obtained, the second film 2 on each of the film assemblies 5 can optionally be removed. In a preferred embodiment, the second film 2 is removed by peeling it off from the first film 1. Preferably, the second film 2 is removed after the curing and machining steps. In another embodiment, the second film 2 is retained during shipping of the shaped article 100 to a desired location, after which it can be removed as discussed above.

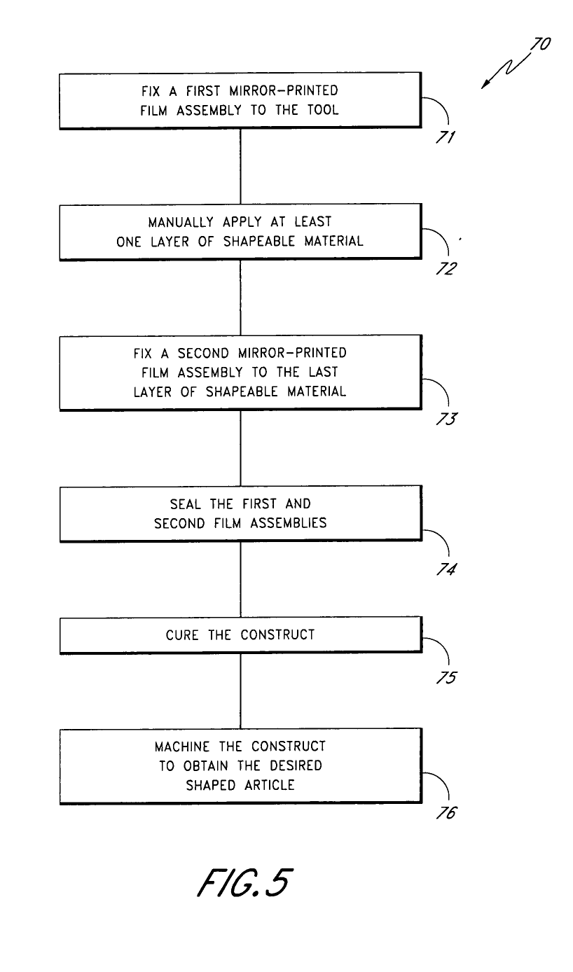

Another embodiment of a method 70 for manufacturing a shaped article with a mirror-printed film thereon is detailed in FIG. 5. This method is similar to the one depicted in FIG. 4, however the lay-up sequence is performed manually 72. The remaining steps 71, 73-76 of the method 70 correspond to steps 61, 63-66 of the method 60 shown in FIG. 4.

In still another embodiment, the lay-up sequence can be performed using a combination of the automatic and manual methods discussed above. For example, where the lay-up sequence includes adding a layer L of shapeable material 40, such as cloth, that must be positioned manually, said layer L can be manually added and the automatic lay-up sequence resumed thereafter.

FIGS. 6A-6G illustrate some of the steps in one embodiment of a method using a combination of automatic and manual methods to apply a mirror-printed film during the manufacture of a shaped article. As shown in FIG. 6A, the layers L of shapeable material 40 to be applied manually are first prepared. For example, the layers may initially be cut to the desired size. A film assembly 5 is first placed on a generally planar surface (not shown) such that the second film 2 faces the planar surface and the bottom surface 1b of the first film 1 faces away from the planar surface, as seen in FIG. 6B. A layer L1 of shapeable material 40 is then placed on top of the first film 1, as shown in FIG. 6C. In one embodiment, the layer L1 of shapeable material 40 comprises carbon cloth. In the illustrated embodiment, the layer L1 of shapeable material 40 includes a protective layer 42 that faces away from the planar surface. FIG. 6D shows the layer L1 of shapeable material 40 following removal of the protective layer 42.

A second layer L2 of shapeable material 40 is applied to at least a portion of the first layer L1 of shapeable material 40, as shown in FIG. 6E. In the illustrated embodiment, the second layer L2 of shapeable material 40 has fibers oriented at 90 degrees to each other and has a protective layer 44 that faces away from the planar surface. In the illustrated embodiment, the protective layer 44 is made of a wax material. FIG. 6F shows the second layer L2 of shapeable material 40 following removal of the protective layer 44. In the illustrated embodiment, the second layer L2 is configured to provide additional structural reinforcement or support in a desired area. Accordingly, the second layer L2 is smaller than the first layer L1. In another embodiment, the second layer L2 can be the same size as the first layer L1. In one embodiment, a protective cover 46 may be temporarily placed over layers L1, L2 of the shapeable material 40 while in storage, as shown in FIG. 6G. More layers L can be added, as desired, in the same manner described above.

The layers L of shapeable material 40 and film assembly 5 can then be placed in a compartment (not shown) and a vacuum applied to remove air from between the layers L and film assembly 5. The protective cover 46 can optionally be removed prior to the application of the vacuum. The assembly consisting of the layers L of shapeable material 40 and film assembly 5 can then be placed on a tool having a desired curved surface, as discussed above, and a machine used to automatically add additional layers of shapeable material. An additional film assembly 5 can be added following the last layer of shapeable material, as discussed above. The shaped article 100 can then be completed in the manner previously discussed.

FIG. 7 illustrates a cross-sectional schematic view of the completed construct 50 manufactured according to one of the methods discussed above. As discussed above, the film assemblies 5 are oriented relative to the construct of shapeable material such that the at least one graphic image 7 is positioned adjacent a layer L of shapeable material 40 and the second films 2 face away from the construct 50.

FIG. 8 shows a completed shaped article 100 to which a mirror-printed film 10 is applied during manufacture, in accordance with any one of the methods discussed above. In the illustrated embodiment, the shaped article 100 is a prosthetic device with multiple layers of shapeable material having a curved configuration. Specifically, the shaped article 100 is a foot plate 110 for a prosthetic foot 200. Further information on different prosthetic designs is included in U.S. patent application Ser. No. 10/642,125, filed Aug. 15, 2003, U.S. patent application Ser. No. 10/674,736, filed Sep. 30, 2003, U.S. patent application Ser. No. 10/742,455, filed Dec. 18, 2003, and U.S. patent application Ser. No. 10/944,436, filed Sep. 17, 2004, the contents of all of which are hereby incorporated by reference and should be considered a part of this specification.

In the illustrated embodiment, the foot plate 110 has an upper portion 112 and a lower portion 114. As shown in FIG. 8, the upper portion 112 of the foot plate 110 is horizontally oriented. In another embodiment, the upper portion 112 of the foot plate 110 can be vertically oriented. At least a portion of the foot plate 110 extends generally downward and forward. The foot plate 110 also has a first surface 116 and a second surface 118, wherein the surfaces 116, 118 are opposite one another. In the illustrated embodiment, the film 10 with a mirror-printed image 7 has been adhered to the first surface 116, using the process described above. In another embodiment another film, with or without a mirror-printed image 7 thereon, can also be adhered to the second surface 118.

As shown in FIG. 8, the foot plate 110 has generally a J-shape and a C-shape. In another embodiment, the foot plate 110 can have a J-shape or a C-shape. However, the foot plate 110 can have other curved shapes, as discussed below.

In another embodiment, the shaped article 100 can be a prosthetic device having one or more foot plates, such as the foot plate 110 described above. Preferably, one or more of the foot plates can have an upper portion at about a location along a natural human lower leg. For example, in one embodiment the upper portion can be at about the location of a natural human ankle. In another embodiment, the upper portion can be at about the location of a natural human shin. Additionally, one or more of the foot plates can have a lower portion at about a location along a natural human sole. For example, in one embodiment, the lower portion can be at about the location of a natural human heel. In another embodiment, the lower portion can be at about the location of a natural human forefoot. In one embodiment, the prosthetic device has first and second foot plates connected to each other, each foot plate having opposing surfaces and constructed from a plurality of fiber-reinforced layers, as discussed above. In another embodiment, the first foot plate has the upper portion and the second foot plate has the lower portion. In one embodiment, the mirror-printed image 7 is provided to the film on at least one of the opposing surfaces of the device. In another embodiment, the mirror-printed film 10 is adhered to each of the opposing surfaces.

The prosthetic device preferably has an attachment portion that operably connects the prosthetic device to a limb. In the embodiment shown in FIG. 8, the attachment portion of the prosthetic foot 200 is an aperture 220 formed at the upper portion 112 of the foot plate 110. The aperture 220 can be formed using a cutting tool, such as a drill or a water jet, as discussed before. Preferably, the aperture is formed while the second film 2 is adhered to the first film 1 on the upper portion 112 of the foot plate 110.

In one embodiment, an adapter can be attached to the prosthetic device. For example, the adapter can be attached to the prosthetic foot 200 via the aperture 220 to operably connect the prosthetic foot 200 to a limb. The adapter preferably has a pyramid connection that connects to a socket or intermediate prosthesis member. For example, the pyramid can receive a pylon or lower leg prosthesis thereon. Preferably, the pyramid extends along an axis generally perpendicular to a surface on which the prosthetic foot 200 sits at rest. In one embodiment, the adapter has a base that generally conforms to, or sits adjacent, the upper portion 112 of the foot plate 110. In another embodiment, the adapter has at least one portion that can “roll-up” or move relative to the foot plate 110 when the foot plate 110 flexes. The adapter is preferably constructed of metal. In one embodiment, the adapter is constructed of titanium and/or aluminum.

In another embodiment, the adapter can have a tube clamp with a generally cylindrical body configured to receive a pylon or other prosthesis therein. For example, the tube clamp can have clamp arms that are urged toward one another to fasten the tube clamp about a surface of the pylon or other prosthesis.

In still another embodiment, the adapter can attach to a shock module having support members that are coaxially aligned and telescopingly engaged with each other. The shock module preferably compresses to store energy during, for example, a heel-strike position of the prosthetic foot 200 during a gait cycle of normal ambulation. Then, as the user's weight shifts closer to a toe-off position, the shock module expands and releases the stored energy, providing beneficial lift and thrust forces to the user.

In one embodiment, a foot cover can be provided, wherein the foot cover surrounds the prosthetic foot 200 or other prosthetic device. In one embodiment, the foot cover can be generally shaped like a human foot.

Additional prosthetic foot designs to which the methods described above can be applied include the following models by Össur of Rekjavik, Iceland: Axia™, Ceterus™, Elation™, LP Ceterus™, LP Vari-Flex™, Modular III™, Re-Flex VSP™, Cheetah™, Flex-Sprint™, Flex-Run™, Talux®, Vari-Flexφ, and Flex-Foot® Junior. However, as previously discussed, the mirror-printed film 10 can be applied to other shaped articles or devices, such as other prosthetic and orthotic devices that approximate a joint of a limb, such as a lower leg, an ankle, and a foot. For example, the mirror-printed film 10 can be applied to a curved device that curves between a generally vertical portion and generally horizontal portion, said portions generally conforming to a J-shape. In another example, the generally vertical and generally horizontal portions may generally conform to an L-shape. The mirror-printed film 10 can also be applied to a curved device having at least one arcuate portion. For example, the device can curve between a first arcuate portion and a second arcuate portion, said first and second portions generally conforming to a C-shape. In another example, the first and second arcuate portions can generally conform to a U-shape. In still another example, the device can have only one arcuate portion extending from a first point to a second point, the first point being at a higher vertical position than the second point. In yet another example, the device can have an arcuate portion that transitions to a generally planar portion. In still another example, the mirror-printed film can be applied to a curved device with multiple curves. For example, the device can have a curved shape generally conforming to an S-shape or a Z-shape.

As shown in FIG. 8, use of the mirror-printed film 10 allows the shaped article to have any desirable graphic(s) 7 applied thereto, and provides the shaped article with a glossy or shiny finish. However, the film can have other suitable finishes. For example, in another embodiment, the mirror-printed film can have a matte finish.

Use of the mirror-printed film 10, as discussed above with respect to FIGS. 1-8, advantageously provides a graphic 7 that is substantially protected from being scratched, as the graphic 7 is printed on the bottom surface 1b of the first film 1 (See FIG. 1). Use of the first film 1 also advantageously dampens vibrations in the shaped article 100 that are generated when the article 100 is used. Dampening of vibrations is particularly advantageous in prosthetic and orthotic devices because vibrations cause discomfort to the user of the device during, for example, walking. Additionally, vibration can cause wear and tear in the prosthetic or orthotic device, resulting in a shorter lifetime for the device.

Use of the mirror-printed film 10 also advantageously provides a shaped article 100 with edges that are less sharp, resulting in less wear and tear to covers used in conjunction with the shaped article. For example, where the shaped article is the foot plate 110 of a prosthetic foot 200, as shown in FIG. 8, a foot cover (not shown) used to enclose the foot plate 110 will experience less wear and tear from contact with the edges of the foot plate.

Use of said mirror-printed film 10 during the manufacture of the shaped article 100 also advantageously simplifies the manufacturing process of the article. For example, use of the mirror-printed film 10 makes it unnecessary to apply a protective layer to the construct 50 before machining the construct 50 into the desired shape of the shaped article 100.

Use of the mirror-printed film 10 also advantageously reduces the amount of material necessary to manufacture the desired shaped device and improves the quality of the shaped article 100. For example, in contrast to traditional methods where a glossy appearance is provided by attaching a stiff glossy metal plate to the construct, a glossy finish can be advantageously provided by the first film 1, without the need to use said metal plates. Additionally, a shaped article 100 having a curved shape that is manufactured using the mirror-printed films 10 in the manner discussed above advantageously has more uniformly laid panels of shapeable material 40. In contrast, use of stiff metal plates to manufacture a shaped article having a curved shape results in a shaped article having less uniformly laid panels of shapeable material 40 because air voids remain in the construct following the curing process. That is, the relative inflexibility of the metal plates prevents substantially all air voids from being removed from the construct during the curing process.

Additionally, the use of the second film 2 in conjunction with the first film 1 provides additional protection to the graphic image 7 and the first film 1. The second film 2 can advantageously be removed after the shaped article 100 is manufactured to ensure the first film 1 and graphic 7 are substantially protected during the manufacturing process.

The various devices, methods and techniques described above provide a number of ways to carry out the invention. Of course, it is to be understood that not necessarily all objectives or advantages described may be achieved in accordance with any particular embodiment described herein. Also, although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein.

What is claimed is:

1. A device for attaching to a limb, comprising:
multiple layers of shapeable material having a curved configuration, said multiple layers together defining a first and second surface of said device; and
a film having at least one mirror-printed image thereon adhered to at least one of said first and second surfaces of the device.

2. The device of claim 1, further comprising an attachment portion configured to operably connect the device to a limb.

3. The device of claim 1, wherein a film is adhered to both the first and second surfaces of the device.

4. The device of claim 3, wherein a mirror-printed image is provided to the film on both the first and second surfaces of the device.

5. The device of claim 1, wherein the mirror-printed image is provided on a bottom surface of the film facing at least one of the first and second surfaces.

6. The device of claim 1, wherein the device is a prosthetic device.

7. The device of claim 6, further comprising a pyramid connection configured to connect to a socket or intermediate prosthetic member.

8. The device of claim 6, wherein the device curves from an upper attachment portion to a ground contacting portion.

9. The device of claim 8, in combination with a foot cover surrounding the prosthetic device.

10. The device of claim 1, wherein the device is an orthotic device.

11. The device of claim 1, wherein the shapeable material comprises fiber-reinforced material.

12. The device of claim 1, further comprising a protective film removably covering said film having at least one mirror-printed image thereon.

13. A prosthetic device, comprising:
a foot plate having an upper portion and a lower portion, wherein at least a portion of the foot plate extends generally downward and forward, said foot plate having first and second surfaces;
an adapter provided adjacent the upper portion of the foot plate and configured to attach said foot plate to a pylon or other leg prosthesis; and
a film applied to at least one of the first and second surfaces, the film having a bottom surface adhered to at least one of the upper and lower surfaces and having a mirror-printed image provided thereon facing at least one of the first and second surfaces.

14. The prosthetic device of claim 13, wherein at least a portion of the foot plate curves generally downward and forward.

15. The prosthetic device of claim 13, wherein the foot plate has generally a J-shape.

16. The prosthetic device of claim 13, wherein the foot plate has generally a C-shape.

17. The prosthetic device of claim 13, wherein a film is applied to both the first and second surfaces of the device.

18. The prosthetic device of claim 17, wherein a mirror-printed image is provided to the film on both the first and second surfaces of the device.

19. The prosthetic device of claim 13, wherein the upper portion of the foot plate is generally vertically oriented.

20. The prosthetic device of claim 13, wherein the upper portion of the foot plate is generally horizontally oriented.

21. The prosthetic device of claim 13, wherein the foot plate is constructed from a plurality of layers of shapeable material.

22. The prosthetic device of claim 21, wherein said shapeable material is fiber-reinforced.

23. The prosthetic device of claim 13, further comprising a protective film removably covering said film having at least one mirror-printed image provided thereon.

24. A prosthetic device, comprising:
one or more foot plates, at least one of said one or more foot plates having an upper portion at about a location along a natural human lower leg and at least one of said one or more foot plates having a lower portion at about a location along a natural human sole, said one or more foot plates each having opposing surfaces;
an adapter provided adjacent the upper portion and configured to attach said one or more foot plates to a pylon or other leg prosthesis; and
a film applied to at least one of the opposing surfaces, the film having a bottom surface adhered to at least one of the opposing surfaces and having a mirror-printed image provided thereon facing at least one of the opposing surfaces.

25. The prosthetic device of claim 24, wherein at least a portion of one of said one or more foot plates curves generally downward and forward between the upper portion and the lower portion.

26. The prosthetic device of claim 24, wherein a single foot plate comprises the upper and lower portions.

27. The prosthetic device of claim 24, comprising a first foot plate and a second foot plate connected to one another.

28. The prosthetic device of claim 27, wherein the first foot plate comprises the upper portion and the second foot plate comprises the lower portion.

29. The prosthetic device of claim 24, wherein a film is applied to all of the opposing surfaces of the device.

30. The prosthetic device of claim 29, wherein a mirror-printed image is provided to the film on a plurality of the opposing surfaces of the device.

31. The prosthetic device of claim 24, wherein each of the foot plates is constructed from a plurality of fiber-reinforced layers.

32. The prosthetic device of claim 24, further comprising a protective film removably covering said film having at least one mirror-printed image provided thereon.

33. A method for manufacturing a device that attaches to a limb, comprising:
providing a film having at least one mirror-printed image thereon;
providing a plurality of layers of shapeable material to form a device for attaching to a limb; and
applying said film to at least one of said layers of shapeable material with the mirror-printed image facing the at least one layer.

34. The method of claim 33, further comprising positioning the film adjacent a curved surface, and applying said plurality of layers over said film.

35. The method of claim 34, wherein said film positioned adjacent the curved surface is a first film, and further comprising applying a second film over said plurality of layers, said second film having a mirror-printed image facing the plurality of layers.

36. The method of claim 33, further comprising configuring said layers with said film into a shape suitable for a prosthetic device.

37. The method of claim 33, further comprising configuring said layers with said film into a shape suitable for an orthotic device.

38. The method of claim 33, wherein said film and said layers together form a construct, and further comprising forming the construct into a device for attaching to a limb.

39. The method of claim 38, wherein forming the construct into a device comprises curing the construct.

40. The method of claim 39, further comprising machining the cured construct to form a device configured to attach to a limb.

41. The method of claim 40, wherein machining the construct comprises cutting the construct into multiple shaped articles each having a mirror-printed image thereon.

42. The method of claim 40, wherein the device has a curved shape and wherein the layers of shapeable material are laid substantially uniformly.

43. The method of claim 42, wherein laying the layers of shapeable material substantially uniformly comprises removing substantially all air voids from between the layers of shapeable material.

44. The method of claim 33, further comprising applying a protective film over the film having at least one mirror-printed image thereon.

45. The method of claim 44, further comprising forming the plurality of layers, the film having at least one mirror-printed image thereon and the protective film into a device for attaching to a limb, and removing the protective film after said forming.

46. The method of claim 33, wherein the film has multiple images mirror-printed thereon, and further comprising cutting the film and the layers into multiple devices for attaching to a limb, each device having one of said multiple images thereon.
